(19) United States
(12) Patent Application Publication
(10) Pub. No.: US 2003/0044863 A1
(43) Pub. Date: Mar. 6, 2003

(54) Invasion complex and methods of targeting

(75) Inventor: Samy Ashkar, Boston, MA (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: Children's Medical Center Corporation

(21) Appl. No.: 10/197,725

(22) Filed: Jul. 18, 2002

Related U.S. Application Data
(60) Provisional application No. 60/306,946, filed on Jul. 20, 2001.
(60) Provisional application No. 60/332,652, filed on Nov. 16, 2001.
(60) Provisional application No. 60/382,794, filed on May 22, 2002.

(51) Int. Cl. G01N 33/574

(52) U.S. Cl. 435/7.23

(57) ABSTRACT

Therapeutics have been identified and developed with are targeted to inhibiting metastasis. These are based on the discovery of an invasion complex which confers the ability of cells, for example tumor cells, to translocate across extracellular matrix barriers, as well as the identification of novel peptides that interact with the invasion complex and regulate it's activity. Whole or partial complexes, or individual molecules of the invasion complex are used to screen for proteins or compounds that interact with the invasion complex. Methods of screening for interacting proteins such as osteopontin, sophin B (SEQ ID NO: 1), or compounds are well known in the art, some of which are described below. Once interacting proteins have been identified, they are screened for inhibition, enhancement, or reduction of complex activity. The presence of proteins of the invasion complex on or within cells is indicative of particular diseases or disorders, for example, those characterized by a tumor cell or activated macrophage. Elevated levels of proteins that interact with the invasion complex, such as osteopontin, are also indicative of cancer, and in particular, metastatic cancer. Diagnostic methods, described below, will assist in the identification of subjects having, or at risk of developing a disorder associated with aberrant expression of any of the identified proteins that form the invasion complex. Because this invasion complex confers the ability to translocate across matrix barriers, peptides that bind to the complex play a role in regulating metastasis and/or organ specific homing of cancer cells.

### CROSS REFERENCE TO RELATED APPLICATIONS

Priority is claimed to U.S. Provisional Application Serial No. 60/306,946 filed on Jul. 20, 2001; U.S. Provisional Application Serial No. 60/332,652 filed on Nov. 16, 2001; and U.S. Provisional Application Serial No. 60/382,794 entitled, “Invasion Complex and Method of Targeting”, by Samy Ashkar, filed on May 22, 2002.

### GOVERNMENT SUPPORT

This invention was made with government support from the U.S. Army (Grant Award No. DAMD 17-99-1-9124). Accordingly, the government has certain rights in the invention.

### BACKGROUND OF THE INVENTION

The present invention is generally in the field of therapeutics and diagnostics based on the discovery of an invasion complex involved in metastasis, infection, and inflammation.

Metastasis is the major cause of failure of currently available anti-cancer therapies, yet very few clinical studies target the metastatic process in the development of new therapies. In addition, few drugs in clinical studies specifically target tumors themselves, but rely on the use of chemotherapeutics which preferentially kill the more rapidly replicating tumor cells as compared to normal cells. A major failure at the pre-clinical stage is that of unacceptably low therapeutic indices. Most of the available drugs have high toxicity and low specificity of action. To impact the future of cancer therapy, there is a need for drugs to be specifically targeted, with different mechanisms of action and high therapeutic index.

It is therefore an object of the present invention to provide new diagnostic and therapeutic targets for cancer therapy, and compounds specifically directed to the new targets.

It is a further object of the present invention to provide new targets for therapy of disorders and diseases involving inflammation and infection.

### BRIEF SUMMARY OF THE INVENTION

Therapeutic and diagnostic targets have been identified and developed which are targeted to inhibiting the assembly or activity of an invasion complex. These are based on the discovery of an invasion complex which confers the ability of cells, for example tumor cells, to translocate across extracellular matrix barriers, as well as the identification of peptides that interact with the invasion complex and regulate its activity. The invasion complex is isolated from cells involved in the targeted disease or disorder, using a process such as dissolution in a gentle buffered detergent solution, followed by immunoreaction with an antibody to a component of the invasion complex, such as a beta-1 integrin. The invasion complex is characteristic of cancer cells, inflammation, infection (viral, bacteria and parasitic), and wound healing (for example, in situations involving angiogenesis or abnormal cell proliferation, such as scar formation). Whole or partial complexes, or individual components of the invasion complex are used to screen for proteins or compounds that interact with the invasion complex. Methods of screening for interacting proteins such as osteopontin, sophin B (SEQ ID NO: 1), or other compounds are well known in the art, some of which are described below. Once interacting proteins have been identified, they are screened for inhibition, enhancement, or reduction of complex activity.

The presence of proteins of the invasion complex on or within cells is indicative of a particular disorder, for example, cancer (a tumor cell) or inflammation (activated macrophages). Elevated levels of proteins that interact with the invasion complex, such as osteopontin, are also indicative of cancer, and in particular, metastatic cancer. Diagnostic methods based on measurements of the invasion complex, components thereof, or molecules which interact with the invasion complex, can be used in the identification of subjects having, or at risk of developing, a disorder associated with aberrant expression of any of the identified proteins that form the invasion complex. Because this invasion complex confers the ability to translocate across matrix barriers, peptides that bind to the complex can be used to inhibit metastasis and/or organ specific homing of cancer cells, inflammation, infection, or modify wound healing.

### DETAILED DESCRIPTION OF THE INVENTION

The Invasion Complex

An invasion complex that is characteristic of cancer, inflammation, infection, and certain wound healing situations, has been isolated and characterized. The complex is obtained by dissolution of the complex from the surface of cells involved in the disease or disorder (for example, tumor cells, inflammatory cells such as macrophages, infected cells) using a buffered solution of a very gentle detergent, followed by binding of the complex to an antibody to the complex or a component thereof, such as beta-1 integrin, as described in the examples. Components of the complex are identified by electropheresis of the complex to separate the different components, of which there are at least twenty-two, which are then identified by immunoreaction with antibodies to known proteins, or isolation and sequence analysis with comparison to known protein data bases.

Although the invasion complex referred to herein has been isolated and identified as described in the examples, not all of the components of the invasion complex have been identified. As described in the examples, components have been identified, and others can be identified, using routine techniques, since the invasion complex in its entirety has been isolated. Components are separated using standard methods such as acrylamide gel electropheresis. The separated components can be identified by reaction with antibodies to known proteins, antibodies to the complex as a whole, or isolation and sequencing of the amino acids of all or part of the protein, which can then be entered into a data base to identify the protein. Components known at this time include CD44(v3-v6), Integrin B1, P13 Kinase, SHP-1, Integrin Associated Kinase, focal adhesion kinase (FAK), Caveolin, tissue plasminogen activator, urokinase plasminogen activator, matrix metalloproteinase 2, matrix metalloproteinase 9, mbMMP (membrane bound metalloproteinase), PKC β, PKC δ, paxillin, rhoB, and G₁I.

CD44 is a transmembrane glycoprotein which normally functions in cell-matrix and cell-cell adhesion interactions, lymphocyte activation and homing, and cell migration. CD44 exists in several isoforms with varying extracellular regions. CD44v3-v6 and other splice variants of CD44 form functional CD44 receptor complexes. These complexes mediate invasion, homing and implantation of metastatic tumor cells. For example, transfection of CD 44 (v6) into benign tumors was sufficient to confer metastatic behavior (Zoller, 1995, J. of Mol. Med. 73, 453-38). Expression of specific variant exons of CD44 on the cell surface of certain tumors has been linked to their metastasis. For example, expression of exon v10 on the surface of melanomas is linked to the metastasis of melanoma cells to skin, while the expression of CD44 (v5) directs melonomas to the lymph nodes. Expression of CD44 (v9) is linked to the metastasis of kidney carcinoma, exon v7-v8 in metastasis of carcinoma of the cervix uteri, and exon v4 and v5 in the metastasis of hepatocellular carcinoma metastasis (Zoller, 1995, J. of Mol. Med. 73, 453-38). The expression of CD44 has also been linked in the metastasis of breast tumors. The expression of CD44 splice variants v3/v4 and v6 correlates with the grading of breast tumors and the expression of CD44 (v6) correlates with metastatic potential of breast tumor and with poor prognosis (Rev. Fichtner et al., 1997, Anticancer Res. 17:5A 3633-45).

The coordination of activities resulting in metastasis is mediated by the unique complex of molecules (CD44 splice variants included) that form the invasion complex. This complex also links outside signaling to inside signaling and functions as a receptor. These invasion complexes not only regulate cell adhesion, mobility and limited extracellular proteolysis, but also link these events with inhibition of apoptosis and the inhibition of proliferation.

FAK is a non-receptor tyrosine kinase that plays an important role in normal cellular processes such as adhesion, spreading, migration, proliferation and survival. In addition, FAK is overexpressed in a variety of cancer cells and tumors, and may play a role in the development of human cancer. Caveolin-1 is a major structural component of caveolae, which are plasma membrane microdomains implicated in the regulation of intracellular signaling pathways. Previous in vitro and in vivo studies on the function of caveolin-1 in carcinoma showed controversial results, indicating that the physiological role of caveolin-1 varies according to the origin of carcinoma. The other components of the invasion complex have also been linked to cancer, although in some cases as inhibitors and in other cases as potentially interactive components.

It is well understood in the art that protein homologs and other proteins with similar activity exist across genus and specie boundaries. Accordingly, the invasion complex described herein will be understood to include complexes of proteins homologous to the component proteins of the complex as disclosed as well as protein complexes with similar invasion function.

Invasion complexes are involved in normal and abnormal cell proliferation, migration and homing (for example, cancer cell metastasis and replication). Based upon the invasion complex's involvement in migration, the invasion complex is a signature of a tumor cell with the ability to invade other tissues (metastasis), as well as invade specific organs (organ specific homing). The invasion complex also modulates chemotaxis, migration, and other modes of cellular recruitment and motility. For example, egg fertilization may be inhibited or enhanced by proteins on the cell surface and their interactions with the donor cell invasion complex. Invasion complexes have also been shown to play prominent roles in other cellular activities such as regulating actin and microfilament rearrangements (playing a critical role in pseudopod formation), as well as shutting down DNA synthesis and replication. The inhibition of DNA replication has a direct impact on cell viability, in many cases inducing apoptosis.

The composition of the invasion complexes is in part dependent on the source of the invasion complex. For example, the transformation of a cell from a non-invasive form to an invasive form is, in part, also related to the presence of specific types of molecules in the invasion complex. Metastatic tumor cells express an excess of β1 integrins over the β5 integrins that are normally present in macrophages. The ratio of β1 integrins to β5 integrins is higher in metastatic tumor cells as compared to macrophages. In addition, CD44 is heparin sulfated in invasion complexes of metastatic tumor cells, contributing to the invasive property of the tumor cell. The CD44 of macrophages is modified by chondroitin sulfate. A shift in the sulfate modification of macrophages, from chondroitin to heparin, can be used in the diagnosis of early phase arthritis. Therefore, the invasion complex can be used, for example, as a marker for arthritis, osteoporosis, and chronic inflammatory diseases depending on the cell type expressing the complex. This is also evidence that the presence of heparin in the invasion complex plays an important role in conferring a migration/invasive phenotype on the cell.

The invasion complex's repertoire of activities includes protease functions that allow the cell to move through the extracellular matrix (ECM). CD44 binds to protease proenzymes (MMPs—matrix metalloproteases) that are subsequently activated to their enzymatic active form by specific proteolytic enzymes (mb MMPs—membrane bound metalloproteases). Once activated, these MMP proteases play a critical role in “carving” out a path through the ECM and tissue for the cell to migrate. As the cell is migrating, pseudopod formation allows for these proteases to form at the forward tip of the cell. The invasion complexes, therefore, play a critical role in the induction of proteases that are assembled at, and associated with, the laminal protruding pseudopod of migratory cells (for example, metalloproteases and plasminogen activators). The types of proteases being expressed often depends upon the type of cell. Once the cell adheres to a target, the cycle starts over with the proteolytic processing of the matrix metalloproteases.

Invasion Complex Molecules/Drug Targets and Associated Diseases

The invasion complex is not just found in metastatic cancer. Diseases that rely upon cell migration and/or cell translocation are also dependent upon the invasion complex. The cell type, and specific components of the invasion complex present on the cell, will dictate the type of disease or cellular process associated with the cellular migration/translocation phenotype. Specific proteins may be part of an invasion complex on one cell type, but not part of an invasion complex on another cell type. This represents a diversity in structure of the invasion complex that may “encode organ specificity in homing and metastasis formation (a “postal code” of sorts)”; (see Weber and Ashkar, J. Mol. Med., 2000, 78:404-408). Diseases relying on cell type and the invasion complex include, but are not limited to, cancer, inflammation (acute and chronic), viral and parasitic infection, wound healing and scarring.

Targets for the disruption of invasion complex formation or activation include components of the invasion complex, or molecules that interact with the invasion complex. For example, CD11 is an important protein in inflammatory bowel syndrome. This molecule may be part of the invasion complex on the surfaces of cells responsible for the inflammatory characteristics of the disease, or may be present on cells that interact with the invasion complex of other cells. Disrupting the interaction between CD11 and the complex as a whole (as presented on two separate cells) or between CD11 and a component(s) of the complex (in order to interrupt the complete formation of the invasion complex), would effectively block cell migration and reduce the inflammation associated with the disease. Other targets include, but are not limited to: β1, β2 (inflammatory myopathy); α5β1 (foot and mouth disease); α2β1 (wound healing, plaque formation); and β1 and β3 (hemorrhagic fever with renal syndrome) (also see Table 1).

TABLE 1
Invasion Complex Molecules/Interacting Molecules and
Associated Diseases.

| Target Molecule | Disease |
|---|---|
| CD11 | Inflammatory Bowel Syndrom |
| β3 | Restenosis and Atherosclerosis |
| α2β1 | Breast Cancer/Cancer |
| β2 | Chronic obstructive pulmonary disease, |
| | cystic fibrosis, adult respiratory distress |
| | syndrome |
| α8β1 | Kidney/cystic disease |
| αvβ3 | Breast Cancer |
| CD31 | Graft rejection |
| β1 | Chronic Myelogeous Leukemia |
| α4 and β7 | MS-like disease: experimental autoimmune |
| | encephalomyelitis |
| β2 | Leukemia-lymphoma and Hodgkins |
| α2b1 | Wound healing, plaque formation |
| αβ | Hemostasis, thrombosis, cancer |
| αvβ2 and a5b1 | Lyme disease |
| αMβ2 (Mac1) | Autoimmune disease, MS |
| LFA-1 | Rheumatoid arthritis, asthma |
| LFA-1 and Mac1 | Leukocyte adhesion deficiency |
| β2 | Graves disease |
| α4β1 | Retinal development |
| β1, β2 | Inflammatory Myopathy |
| α5β1 | Foot and mouth disease |
| β4 | Junctional Epidermolysis bullosa with |
| | pyloric atresia |
| β1 and β3 | Hemorrhagic fever with renal syndrome |
| GP IIa/IIIb | Acute coronary syndromes |
| α2β1 | Type 1 von Willebrand disease |

Targets also include mechanisms driving the assembly of the invasion complex, cell(invasion complex)-cell interactions, cell(invasion complex)-matrix interactions, activation of MMPs (matrix metalloproteases), inhibition of apoptosis, and inhibition of DNA replication (and proliferation).

The invasion complex confers to cells, for example tumor cells, the ability to translocate across extracellular matrix barriers as well as the identification of novel peptides that interact with the invasion complex and regulate its activity.

Screening for Molecules that Interact with Invasion Complex.

As will be described below, components of the invasion complex may be isolated from cells in which they are expressed. Once whole or partial complexes have been isolated, they may be used to screen for inhibitors or enhancers of complex activity, using the minicell display library technology described in PCT/02/06921 by Childrens Medical Center Corporation. The minicell display technique allows one to generate completely random libraries or libraries based upon derivations of known proteins or peptides.

There are several examples of methods that use peptides or oligonucleotides to develop libraries of potential receptors, enzymes, or antibody interacting peptides. These libraries can be used to uncover proteins or peptides that will interact with the invasion complex, and may therefore be useful as therapeutics. Over the course of the last two decades these libraries have been incorporated into systems that allow the expression of random peptides on the surfaces of different phage or bacteria. Many publications have reported the use of phage display technology to produce and screen libraries of polypeptides for binding to a selected target. Any method that establishes a physical association between DNA encoding a polypeptide to be screened and the target polypeptide or complex may be used to identify peptides that interact with the invasion complex.

Another approach to obtaining peptides that interact with the invasion complex may incorporate the use of native bacterial membranes as carriers for the peptide of interest. Methods are well known in the art, in which the anchoring of proteins on a bacterial surface as a fusion of the desired recombinant polypeptide to a native protein that is normally exposed on the cell's surface.

More recent advances have incorporated the use of fusion proteins comprising pilin proteins such as (TraA) or a portion thereof and a heterologous polypeptide displaying the library peptide on the outer surface of the bacterial host cell capable of forming pilus. See U.S. Pat. No. 5,516,637 to Huang et al and the FliTrx™ (Invitrogen Corp.) random peptide library. Methods such as these will aid in the elucidation and isolation of peptides and proteins that interact with invasion complexes and thereby affect downstream biological processes.

Other methods known in the art used to detect protein-protein interactions may be utilized to determine binding and subsequent isolation of interacting peptides. Such methods include, for example, co-immunoprecipitation assays, two-hybrid screens and libraries, and detectable enzymatic reactions that are dependent upon the presence of both receptor (complex) and ligand (peptide).

An example of an invasion complex interacting peptide is the acetylated and non-acetylated forms of Sophin B (SEQ ID NO:1). Sophin B interacts with CD44 and β1 of the invasion complex. This interaction results in the dissociation of the complex.

Screening Interacting Peptides for Activity.

A bioactivity can be any biological effect or function that a peptide or protein may have or exert when interacting with the invasion complex. For example, bioactivities include specific binding to biomolecules (for example, receptor complex ligands), hormonal activity, cytokine activity, and inhibition of biological activity or interactions of other biomolecules (for example, agonists and antagonists of receptor binding), enzymatic activity, anticancer activity, immunosuppressive activity, immunostimulatory activity, immune characteristic, alteration of the function of immune system cells, antibiotic activity, antiviral activity, and trophic activity. Bioactivity can be detected and/or measured using appropriate techniques and assays known in the art. Antibody reactivity and T cell activation can be considered bioactivities. Bioactivity can also be assessed in vivo where appropriate. This can be the most accurate assessment of the presence of a useful level of the bioactivity of interest. Enzymatic activity can be measured and detected using appropriate techniques and assays known in the art.

Several peptides that bind to the invasion complex and are required for tumor metastasis have been identified. Several have been identified that block metastasis. These metastasis blocking peptides have been further evaluated for effecting a particular response on the receptor that can be assayed biochemically. Peptides have been shown to influence the autophosphorylation of receptors in vitro, by assaying the amount of radiolabeled phosphate retained by the receptor before and after interaction with the peptide. This can be shown using standard techniques within the field of molecular biology. By influencing the phosphorylation of cell surface receptors, the isolated peptides directly influence the activity of the cellular processes these receptors control.

Peptides that Bind to Invasion Complex and Affect Cell Viability.

Further in vitro analyses are used to study the effects of the invasion complex binding peptides and their effects on cell viability. Peptides that either interrupt, stimulate, or decrease vital cellular processes may be used to infect cells, such as tumor cells, in culture. Once infected, cell growth and viability is analyzed by methods known in the art.

Many cells may undergo programmed cell death which is a genetically mediated form of self destruction. This phenomenon is commonly referred to as apoptosis. Cells that undergo apoptosis due to the peptides interacting with this invasion complex may be analyzed before, during, and after apoptosis. Frequently, apoptotic cells may be recognized by changes in their biochemical, morphological and molecular features. Morphological changes include but are not limited to cell shape change, cell shrinkage, cell detachment, apoptotic bodies, nuclear fragmentation, nuclear envelope changes and loss of cell surface structures. Biochemical changes may include proteolysis, protein cross linking, DNA denaturation, cell dehydration, intranucleosomal cleavage and a rise in free calcium ions.

When cells are no longer viable (dead), their membranes become permeabilized and this permeabilization will manifest itself as a change in the scattering of light. This scattering of light can be attributed to the change in the refractive index of the cell's cytoplasm. The use of DNA staining dyes that are able to pass through a permeabilized membrane, will aid in the identification of dead, live, and apoptotic cells. Flow cytometry and/or fluorescent activated cell sorting (FACS analysis) may be incorporated into protocols utilizing fluorescent dyes to separate the cells of interest. Flow cytometry can sort, or physically separate, particles of interest from a sample. Therefore, FACS analysis (which is a type of flow cytometry), may be defined as the physical separation of a cell or particle of interest from a heterogeneous population.

One may distinguish between dead, live, and apoptotic cells because each differ, for example, in their permeability to DNA dyes. Two widely used DNA dyes, Hoechst 33342 and propidium iodide (PI), are able to infiltrate dead cells. Live cells do not retain either dye, while apoptotic cells retain Hoechst but not PI. Fluorescent microscopic observation allows one to visually separate dead cells from live cells from cells undergoing apoptosis. Fluorescence emission from these different cells allows their separation via flow cytometry and/or FACS analyis. Typical stains used in these assays will include, propidium iodide, Hoechst 33342, 7AAD and TO-PRO-3.

Stages of membrane change during apoptosis may be analyzed as well. Among these changes is the translocation of phosphatidylserine (PS) from the inner part of the cell membrane to the outside during the early to intermediate stages of apoptosis. Using FITC labeled Annexin V, one may be able to detect PS. Annexin V is a Ca⁺⁺ dependent phospholipid-binding protein. Again, dead cells will not bind Annexin V. Live cells are also negative for Annexin Binding. Apoptotic cells bind Annexin. One may combine this method of analyzing PS with the aforementioned method of using PI to stain DNA, thereby obtaining different profiles of live, dead, and/or apoptotic cells.

As mentioned above, a characteristic of apoptosis is the degradation of DNA. This degradation is usually carried out by activated Ca/Mg dependent endonucleases. Terminal deoxynucleotidyl transferase (TdT) will add biotinylated, BrdU or digoxygenin-labeled nucleotides to DNA strand breaks. Subsequent binding of the exogenously added streptavidin by the biotin, or a fluorochrome labeled anti-digoxygenin antibody may be used to then detect DNA degradation. This method allows one to correlate apoptosis with cell cycle status.

Another DNA binding dye that may be incorporated is laser dye styryl-751 (LDS-751). Again, one may take advantage of the ability of apoptotic cells to exhibit different staining patterns than that of live or dead cells.

Laser capture micro-dissection (LCM) is a relatively new technology used for the procurement of pure cells from various tissues. After transfer film is applied to the surface of a particular tissue section, one may activate a pulsed laser beam that, in turn, activates the film immediately above the cell(s) of interest. The film melts and fuses the underlying cells. The film can then be removed and the remaining cells, not contained within the film, are left behind. Once the cells are isolated, DNA, RNA or protein from the cells may then be purified. The isolation of the cells via LCM does not damage the cells because the laser energy is absorbed by the film. This particular technology may be useful in combination with any of the previously mentioned methods of detecting proteins using fluorescent molecules.

In vivo analyses using animal models is used to determine the effects of the peptide within an intact system. For example, in the field of immunology, peptides can be administered to an animal and its peripheral blood monocytes are used in the generation of antibodies directed against the peptide.

In the case of viral proteins (for use with, for example, viral vectors, therapeutic viruses, and viral capsid delivery compositions) desired characteristics to be retained by the peptide can include the ability to assemble into a viral particle or capsid and the ability to infect or enter cells. Such characteristics are useful where the delivery properties of the viral proteins are of interest. The delivery of the peptide into the cell could then interrupt formation of the complex.

Prognostic Assays.

The proteins of the invasion complex are expressed, for example, in activated macrophages and diseased cells, such as tumor cells. Using these examples, the presence of any of the proteins that form the complex, proteins associated with the formed complex, or any transcripts indicating expression of the genes encoding the proteins, is an indicator of the presence of tumor cells and/or activated macrophages. An example of a protein which interacts with, but is not part of, the invasion complex, is osteopontin, which has now been shown to be associated with invasive breast cancer and certain other types of cancer.

The diagnostic methods described herein can be further utilized to identify animals or humans having or at risk of developing a disease or disorder associated with invasion complex formation. The assays can be utilized to identify a subject having or at risk of developing a disorder associated with aberrant expression of any of the herein identified proteins that form the invasion complex (CD44(v3-v6), Integrin β1, PI3, KinaseSHP-1, Integrin Associated Kinase, FAK, Caveolin, tissue plasminogen activator, urokinase plasminogen activator, matrix metalloproteinase 2, matrix metalloproteinase 9, mbMMP (membrane bound metalloproteinase), PKC β, PKC 6, paxillin, rhoB, and the G-protein G₁I).

In preferred embodiments, the methods include detecting, in a sample of cells from a subject, the presence or absence of a genetic alteration/mutation in a gene or regulatory sequence affecting the expression of any of the proteins that form the complex and/or associated with the complex and its formation. For example, such alterations/mutations can be detected by ascertaining the existence of at least one of: 1) a deletion of one or more nucleotides from a gene or regulatory sequence that regulates the expression of any of the genes encoding proteins of the complex; 2) an addition of one or more nucleotides to a gene or regulatory sequence that regulates the expression of any of the genes encoding proteins of the complex; 3) a substitution of one or more nucleotides from a gene or regulatory sequence that regulates the expression of any of the genes encoding proteins of the complex; 4) a chromosomal rearrangement of a gene or regulatory sequence that regulates the expression of any of the genes encoding proteins of the complex; 5) an alteration of the level of mRNA of any of the transcripts encoding any of the proteins of the invasion complex; 6) aberrant modification of a gene encoding any of the protein components of the complex or any protein required for the formation of the complex; 7) the presence of an abnormal splicing pattern of a messenger RNA transcript of any of the genes encoding proteins of the complex; 8) an aberrant level of any or all of the proteins of the complex; and 9) inappropriate post-translational modification of a protein of the invasion complex. There are a large number of techniques known in the art which may be used to detect alterations in any of the genes encoding the proteins listed above.

Examples of assays that are used to detect genetic alterations/mutations include but are not limited to: PCR, RACE PCR, LCR (ligation chain reaction), nucleotide arrays (genomic or oligo) of DNA or RNA to be used in hybridization assays, alternative restriction enzyme digestion patterns, direct sequencing, mismatch cleavage assays of nucleic acid duplexes, electrophoresis and/or polyacrylamide electrophoresis, Northern and Southern Blot assays, RNA primer extension.

In one embodiment, the presence of proteins or transcripts encoding the proteins of the complex in a biological sample is a marker that determines the presence of cancerous cells in the sample.

In another embodiment, the presence of proteins or transcripts encoding the proteins of the complex in a biological sample is a marker that determines the presence of metastatic cancerous cells in the sample or cancerous cells with the potential to become metastatic:

The following examples are offered by way of illustration an not by way of limitation.

### EXAMPLE 1

### Metastatic Invasion of Tumor Cells in vivo

Nude mice were anesthetized by injection with Ketamin/xylazine, 90 mg/10 mg mixed together with sterile H₂O or saline, 20 g, IP, then scrubbed for aseptic surgery. A 30-gauge needle mounted onto a tuberculin syringe was inserted into the second intercostal space, 2 mm to the left of the sternum and aimed towards the heart. The entrance of bright red blood into the syringe indicates proper positioning of the needle in the left ventricle of the heart. 2×10 tumor cells are then injected into the left ventricle of nude mice. The mice are allowed to recover from anesthesia. All mice were evaluated 56 days after tumor cell injection.

The tumor cells injected were of three groups:

(a) CD44⁻, β3⁻ or β5⁻, β1+, TPA/UPA⁺, mmp⁻;

(b) CD44⁺, β3⁻ or β5⁻, β1⁺; and

(c) CD44⁺, β3⁺ or β5⁺, TPA/UPA⁺.

The tumor cells from group (a) did not result in any tumors. The tumor cells from group (b) resulted in a scattering of tumors (none associated with bone). The tumor cells from group (c) resulted in the formation of bone tumors. This assay confirmed that several receptors are essential for metastasis.

### EXAMPLE 2

### Isolation of Invasion Complex in Tumor Cells (I)

Given these observations, the invasion complex was isolated using benign human breast cancer tumor cells (MB-453) transfected with CD44. Invasion complex components subsequently identified included CD44(v3-v6), Integrin B1, P13 Kinase, SHP-1, Integrin Associated Kinase, FAK, Caveolin, tissue plasminogen activator, urokinase plasminogen activator, matrix metalloproteinase 2, matrix metalloproteinase 9, mbMMP (membrane bound metalloproteinase), PKC β, PKC δ, paxillin, rhoB, and G₁I.

The isolation of the invasion complex is a delicate process that is dependent upon the use of specific reagents. SDS, TWEEN®, TRITON®, and BRIJ® are too harsh to be used to dissociate cells harboring the invasion complex(es). Deoxycholate detergent is too gentle to be used in this process. The amphiphilic “ZWITTERGENT®”, 3-12 or 3-16 (CALBIOCHEM®), is used at pH 7.4 (0.8-1.0%) with phosphate buffered saline, Ca⁺⁺, and Mg⁺⁺ (to stabilize the invasion complex), to dissociate and disrupt the cells.

The invasion complex was immunoprecipitated using anti-β1 integrin antibody and the components were separated on an acrylamide gel. In addition, two-dimensional (2-D) gel electrophoresis was used to identify invasion complex components. This technique separates proteins in terms of their isoelectric points and molecular weights. Two-dimensional electrophoresis is commonly used to identify new cellular components (cytoskeletal proteins, organelle components, etc.) and to detect alterations in their expression using qualitative and quantitative comparisons. Once the proteins are separated via SDS-PAGE, Western analysis may be used to further identify and characterize the components of the complex.

The immunoprecipitation protocol relies upon two buffers: running buffer (2 μl of 1.25 Tris-HCl, pH 6.8, 35 μl distilled water, 2.5 μl 2-mercaptoethanol, 12.5 μl of 10% SDS, 10 μl of 80% glycerol, and 2 μl of bromophenol blue) and TNE buffer (1 mM Tris-HCL, pH 8.0, 10 mM NaCl and 0.5 mM EDTA). Cells are removed from the dissociation solution described above and pelleted in a centrifuge. Cell pellet is resuspended in 1 ml of TNE containing 1% NP40 and vortexed. The suspension is incubated on ice for 30 minutes or at 37° C. for 10 to 15 minutes. Cell debris is pelleted in Eppendorf centrifuge of 3 minutes. The resulting supernatent is transferred to a fresh tube and 8 μl of antisera is added. The resulting solution is incubated on ice for 2 hours or overnight at 4° C. 1001l of Staphylococcus aureus protein A and 100 μl of 5% BSA in TNE is added after incubation. The resulting solution is incubated on ice for 2 hours. The resulting immune complexes are pelleted and washed twice with 1 ml 1%NP40, 0.5% Na deoxycholoate, 0.1% SDS in TNE or ZWITTERGENT° 3-12 or 3-16 (CALBIOCHEM®). The resulting pelleted is resuspended in 70 μl of running buffer and then boiled in water bath for 90 seconds. The resulting solution, in centrifuged for 1 minute to remove the S. aureus (saving the supernatent). The resulting solution is then refrigerated, if the preparation is to be stored before use.

### EXAMPLE 3

### Isolation of Invasion Complex in Tumor Cells (II)

The invasion complex was also isolated using benign human breast cancer tumor cells (MB-453) transfected with CD44. Invasion complex components subsequently identified included CD44(v3-v6), Integrin B 1, P13 Kinase, SHP-1, Integrin Associated Kinase, FAK, Caveolin, tissue plasminogen activator, urokinase plasminogen activator, matrix metalloproteinase 2, matrix metalloproteinase 9, mbMMP (membrane bound metalloproteinase), PKC β, PKC δ, paxillin, rhoB, and G₁I.

The isolation of the invasion complex via cellular dissociation is a delicate process that is dependent upon the use of specific reagents. SDS, Tween, Triton, and Brij are too harsh to be used to dissociate cells harboring the invasion complex(es). Deoxycholate detergent is too gentle to be used in this process. The amphiphilic “ZWITTERGENT®”, 3-12 or 3-16 (CALBIOCHEM®), is used at pH 7.4 (0.8-1.0%) with phosphate buffered saline, Ca++, and Mg++(to stabilize the invasion complex), to dissociate and disrupt the cells.

The invasion complex was isolated from the dissociated cells by affinity column chromatography using phosphorylated osteopontin as the ligand on the column.

### Example 4

### Identification of Invasion Complex Components

³⁵S-labeled invasion complex samples (purified from fresh cultures of either human osteoclasts or breast tumor bone metastases) were suspended in a fresh solution containing 8M urea, 4% (w/v) CHAPS, Tris base 40 mM, DTE 65 mM and a trace of bromophenol blue. A non-linear immobilized pH gradient (3.5-10.0 NL IPG 18 cm) was used as the first dimension. After the first dimension run, the strips were equilibrated in order to resolubilize the proteins and to reduce disulfide bonds. The strips were equilibrated within the strip tray with 100 ml of a solution containing Tris-HCl (50 mM) pH 8.4, urea (6M), glycerol (30% v/v), SDS (2% w/v), iodoacetamide (2.5% w/v) and a trace of Bromophenol blue for 5 minutes.

Those skilled in the art will recognize, or be able to ascertain using no more than routine experimentation, many equivalents to the specific embodiments of the invention described herein. Such equivalents are intended to be encompassed by the following claims.

I claim:

1. A method for identifying cancer in a patient comprising
(a) isolating cells or tissue from the patient; and
(b) identifying an invasion complex, or one or more invasion complex components, associated with the cells or tissue.

2. The method of claim 1 wherein the invasion complex is associated with or identified by an assay for cell migration.

3. The method of claim 1 wherein the invasion complex is associated with or identified by an assay for tumorogenicity.

4. The method of claim 1 wherein the invasion complex comprises a homing component, a proteolytic enzyme and interacts with a ligand, such as osteopontin.

5. The method of claim 1 wherein the invasion complex is obtained by immunoreaction with an antibody to a protein selected from the group consisting of integrin B-1, P 13 kinase, SHP-1, integrin associated kinase, focal adhesion kinase, caveolin, and CD44.

6. The method of claim 5 wherein the invasion complex is obtained by immunoreaction with an antibody to a component selected from the group consisting of integrin β-1, integrin β-3, CD44, and integrin β-5.

7. The method of claim 1 wherein the invasion complex components are over-expressed, underexpressed, or mutated in the cells or tissue as compared to normal cells or tissue.

8. The method of claim 1 wherein the invasion complex components are identified as nucleic acid sequences.

9. The method of claim 8 wherein the nucleic acid sequences encode a protein selected from the group consisting of CD44(v3-v6), Integrin B1, P13 Kinase, SHP-1, Integrin Associated Kinase, focal adhesion kinase (FAK), Caveolin, tissue plasminogen activator, urokinase plasminogen activator, matrix metalloproteinase 2, matrix metalloproteinase 9, mbMMP (membrane bound metalloproteinase), PKC , PKC 6, paxillin, rhoB, and G₁I.

10. The method of claim 1 wherein the invasion complex components are proteins selected from the group consisting of CD44(v3-v6), Integrin B 1, P13 Kinase, SHP-1, Integrin Associated Kinase, focal adhesion kinase (FAK), Caveolin, tissue plasminogen activator, urokinase plasminogen activator, matrix metalloproteinase 2, matrix metalloproteinase 9, mbMMP (membrane bound metalloproteinase), PKC β, PKC δ, paxillin, rhoB, and G₁I.

11. An isolated invasion complex, or portion thereof comprising more than one protein.

12. The invasion complex claim 11 wherein the invasion complex is associated with or identified by an assay for cell migration.

13. The invasion complex of claim 1 wherein the invasion complex is associated with or identified by an assay for tumorogenicity.

14. The invasion complex of claim 11 wherein the invasion complex comprises a homing component, a proteolytic enzyme and interacts with a ligand, such as osteopontin.

15. The invasion complex of claim 11 wherein the invasion complex is obtained by immunoreaction with an antibody to a protein selected from the group consisting of integrin B-1, P13 kinase, SHP-1, integrin associated kinase, focal adhesion kinase, caveolin, and CD44.

16. The invasion complex of claim 15 wherein the invasion complex is obtained by immunoreaction with an antibody to integrin B-1.

17. The invasion complex of claim 11 wherein the invasion complex components are over-expressed, underexpressed, or mutated in the cells or tissue as compared to normal cells or tissue.

18. The invasion complex of claim 11 wherein the invasion complex components are identified as nucleic acid sequences.

19. The invasion complex of claim 18 wherein the nucleic acid sequences encode a protein selected from the group consisting of CD44(v3-v6), Integrin B1, PI3 Kinase, SHP-1, Integrin Associated Kinase, focal adhesion kinase (FAK), Caveolin, tissue plasminogen activator, urokinase plasminogen activator, matrix metalloproteinase 2, matrix metalloproteinase 9, mbMMP (membrane bound metalloproteinase), PKC β, PKC δ, paxillin, rhoB, and G₁l.

20. The invasion complex of claim 11 wherein the invasion complex components are proteins selected from the group consisting of CD44(v3-v6), Integrin B 1, PI3 Kinase, SHP-1, Integrin Associated Kinase, focal adhesion kinase (FAK), Caveolin, tissue plasminogen activator, urokinase plasminogen activator, matrix metalloproteinase 2, matrix metalloproteinase 9, mbMMP (membrane bound metalloproteinase), PKC β, PKC δ, paxillin, rhoB, and G₁I.

21. A method for inhibiting cancer metastasis comprising
providing a compound that inhibits the assembly or an activity of an invasion complex as defined by claim 11, or a component thereof.

22. A method for identifying compounds useful in the diagnosis or inhibition of cancer metastasis, comprising
identifying compounds that interact with an invasion complex or one or more of the proteins of the invasion complex.

23. The method of claim 22 further comprising determining if the compounds alter the assembly or an activity of the invasion complex.

24. The method of claim 22 further comprising determining the levels of compound present in an individual or sample comprising the invasion complex.

25. The method of claim 22 further comprising:
(a) isolating cells or tissue from the individual; and
(b) identifying one or more invasion complex components in the cells or tissue.

26. The method of claim 25 wherein the invasion complex components are identified as nucleic acid sequences.

27. The method of claim 26 wherein the nucleic acid sequences encode a protein selected from the group consisting of CD44(v3-v6), Integrin B 1, PI3 Kinase, SHP-1, Integrin Associated Kinase, focal adhesion kinase (FAK), Caveolin, tissue plasminogen activator, urokinase plasminogen activator, matrix metalloproteinase 2, matrix metalloproteinase 9, mbMMP (membrane bound metalloproteinase), PKC β, PKC δ, paxillin, rhoB, and G₁I.

28. The method of claim 25 wherein the invasion complex components are proteins selected from the group consisting of CD44(v3-v6), Integrin B1, PI3 Kinase, SHP-1, Integrin Associated Kinase, focal adhesion kinase (FAK), Caveolin, tissue plasminogen activator, urokinase plasminogen activator, matrix metalloproteinase 2, matrix metalloproteinase 9, mbMMP (membrane bound metalloproteinase), PKC β, PKC δ, paxillin, rhoB, and G₁I.

29. An isolated compound which interfers with the assembly or an activity of the invasion complex or a component thereof.

30. A diagnostic agent selectively reactive with an invasion complex or an isolated component thereof.

31. The agent of claim 30 wherein the agent is labeled for detection in an assay.
